(19) United States
(12) Patent Application Publication
(10) Pub. No.: US 2008/0050332 A1
(43) Pub. Date: Feb. 28, 2008

(54) Method of skin care and/or treatment using glutaredoxin

(76) Inventor: Hannah Naomi Sivak, Gilbert, AZ (US)

Correspondence Address: Hannah Naomi Sivak, 1213 W Seascape Dr, Gilbert, AZ, 85233, US

(21) Appl. No.: 11/510,166

(22) Filed: Aug. 25, 2006

(51) Int. Cl. A61K 38/44; A61K 8/65

(52) U.S. Cl. 424/70.14; 424/94.4

(57) ABSTRACT

Methods for the prevention and treatment of skin damage arising from the exposure to UV radiation, air pollution and other stressors capable of leading to the formation of free radicals, using the small-dithiol protein glutaredoxin in a dermatologically acceptable carrier that can be applied topically are disclosed. Some embodiments may include other proteins such as superoxide dismutase and/or catalase, as well as other antioxidant molecules such as reduced glutathione, vitamin E, vitamin C, lycopene, astaxanthin and/or tocotrienols.

### FIELD OF THE INVENTION

The present invention generally relates to compositions and methods for preventing skin photo-aging and more particularly to compositions including glutaredoxin and other antioxidants and to methods of using and forming the composition

### BACKGROUND OF THE INVENTION

Free radicals are formed in tissues in multiple ways, for example, as a result of exposure to sunlight. Free radicals initiate chain reactions that produce hydroxyl, superoxide and other radicals, which can damage DNA and other macromolecules. Other processes that generate free radicals are respiration and phagocytosis.

To control the damage done by these free radicals, mammals have a number of antioxidant systems. Some of these antioxidants are obtained from the diet, like vitamin C, vitamin E, carotenoids, and polyphenols; and others are produced endogenously like glutathione and thioredoxin. Antioxidant enzymes, like superoxide dismutases and glutathione peroxidases, are also required.

Insufficient or unsuitable antioxidants may lead to oxidative stress and the consequent oxidation of membrane lipids, damage to DNA, etc. Cellular dysfunctions resulting from oxidative stress include excessive cell proliferation, cell death by apoptosis, lipid deposition, and mutagenesis. By creating such disorders, oxidative stress is partly responsible from a great number of age-related diseases as cancer, cardiovascular disorders, and neurodegenerative diseases as Alzheimer's disease.

Damage by free radicals formed in response to ultraviolet (UV) light is now believed to be a major cause of skin aging, with the term “photo-aging” being used to describe the multitude of effects of sunlight on the skin, including wrinkles, changes in the matrix proteins elastin and collagen, “age” spots (also called sun spots) and an increase in the incidence of skin cancer.

The treatment for sunburn, i.e., erythema in skin caused by solar exposure, used to be the topical use of lubricants and emollients. Thanks to a better understanding of the processes involved, new and more efficient treatments to treat skin damage caused by free radicals have been devised, and they include the topical application and/or ingestion of a variety of natural and synthetic antioxidants. Sometimes, the efficacy of these treatments may be limited because endogenous glutaredoxin may be insufficient. Topical application of glutaredoxin will help alleviate skin problems caused by solar or other types of free radiation producing free radicals, like radiation therapy for cancer patients.

Glutaredoxins are small proteins that operate in thiol-disulfide reactions via two vicinal (CXYC) active site cysteine residues, which either form a disulfide (oxidized form) or a dithiol (reduced form). Glutaredoxins have a molecular mass of approximately 12,000. Animals, plants, and most microorganisms possess a system in which thioredoxin is reduced by the ubiquitous tripeptide glutathione (GSH) and oxidized glutathione (GSSG) in turn is reduced by NADPH in a reaction catalyzed by glutaredoxin.

Reduced glutaredoxins have been found to serve as electron donors in a variety of cellular redox reactions. Glutaredoxins are involved in many physiological processes like cell division, hormone action, transcription, meiosis, DNA replication, protein assembly and repair, and early embryo development. There is also abundant scientific research suggesting an important role of the glutaredoxin system in the control of free radicals in animal tissues. Glutaredoxin is a hydrogen donor for ribonucleotide reductase. It also operates as a general protein disulfide reductase with a specificity for GSH-mixed disulfides. Glutaredoxins have a large number of functions overlapping with thioredoxins but also unique functions related to redox regulations via glutathione. These enzymes may serve as redox sensor and provide protection and recovery from oxidative stress. Glutaredoxin seems to provide antioxidant protection in human coronary arteries and placental function against the oxidative stress caused by pre-eclampsia and may even protect cerebellar granule neurons from dopamine-induced programmed cellular death. Glutaredoxin may also help catalyze the regeneration of ascorbic acid, a water-soluble antioxidant vital to cell function.

### DESCRIPTION OF THE INVENTION

The objective of this invention to provide a method and composition for a preventive regimen and/or therapy based upon the topical application to exposed or affected skin areas of at least one active agent, in association with a dermatologically acceptable carrier or vehicle. This invention is based upon the finding that glutaredoxin is useful for the disarming of active oxidative species. Glutaredoxin may also increase the efficacy of other ingredients in topical compositions for the prevention and treatment of burns.

These and other objectives are accomplished by the present invention, which provides methods and compositions for the prevention and/or treatment of skin damage caused by ultraviolet radiation or other radiations or by any kind of stress leading to the formation of free radicals, by applying topically to the exposed or affected skin areas an effective amount of glutaredoxin, preferably in a dermatologically acceptable carrier.

As used herein, the term “glutaredoxin” encompasses glutaredoxins from any source, plant animal or from microorganisms, purified from natural sources or obtained by genetic engineering means, i.e., by over-expression of a protein, natural or foreign to the organism used for its production.

Glutaredoxins obtained by over-expression in yeast or bacteria are preferred because they are less expensive than those obtained by purification from natural materials.

Many embodiments incorporate at least one other active ingredient with the glutaredoxin. These include reduced glutathione. Other ingredients include natural or synthetic antioxidant molecules such as tocotrienols, vitamin E, ascorbic acid, astaxanthin, and/or lycopene. Other desirable ingredients are proteins capable of alleviating oxidative stress such as catalase and/or superoxide dismutase.

In the preferred practice of the invention, glutaredoxin is applied in admixture with a dermatologically acceptable carrier or vehicle (e.g., as a lotion, cream, ointment, serum) so as to facilitate topical application and, in some cases, provide additional therapeutic effects as might be brought abc it by moisturizing the affected skin areas. As noted, other ingredients, particularly reduced glutathione, are advantageously included in the compositions.

The amount of glutaredoxin necessary to bring about prevention and/or therapeutic treatment of damage by oxidative stress is not fixed, and is dependent upon the source and purity of glutaredoxin employed, the amount and type of any additional ingredients used, particularly those that appear to exhibit synergistic effects, the skin type of the user, and, where present, the severity and extent of skin damage. Generally, the glutaredoxin or composition containing it is topically applied in effective amounts to skin areas which have been damaged, or which are susceptible to damage, because of exposure to a cause of oxidative stress.

In one embodiment, the composition contains from about 0.0001% to about 1% (weight per volume), preferably from more than 0.001% or 0.002% to about 0.01% glutaredoxin.

While the carrier for glutaredoxin can be very simple (such as saline solution), it is generally preferred that the carrier be a composition that will facilitate topical application, and particularly one which will form a film or layer on the skin to which it is applied so as to localize the active ingredient. Many such compositions are known in the art, and can take the form of lotions, creams, gels, etc. Typical compositions include lotions containing water and/or alcohols and emollients such as natural oils and waxes, silicone oils, hyaluronic acid, glyceride derivatives, fatty acids or fatty acid esters or alcohols or alcohol ethers, lanolin and derivatives, polyhydric alcohols or esters, wax esters, sterols, phospholipids and the like, and generally also emulsifiers (nonionic, cationic or anionic), although some of the emollients inherently possess emulsifying properties. These same general ingredients can be formulated into a cream rather than a lotion, or into gels, or into solid sticks by utilization of different proportions of the ingredients and/or by inclusion of thickening agents such as gums or other forms of hydrophilic colloids. Such compositions are referred to herein as dermatologically- acceptable carriers.

Many preferred embodiments of this invention contain at least one or two, and sometimes several, other active ingredients in addition to glutaredoxin, provided that the ingredients are not acids present in concentrations high enough to denature and inactivate glutaredoxin, a protein.

Some embodiments may include reduced glutathione.

Tocotrienol, lycopene, astaxanthin, ascorbic acid, and/or vitamin E may also be added to the glutaredoxin composition, alone or in combination with reduced glutathione in some embodiments.

In terms of a possible explanation for the effectiveness of the active ingredients in the prevention or treatment of damage to the skin, it is noted that glutaredoxin, as part of an antioxidant system, will help scavenge free radicals such as the oxygen radicals created by exposure of skin cells to ultraviolet radiation. Just like in the living cell, where a number of antioxidants work in a concerted fashion, some embodiments of this invention also use the synergistic effect of antioxidants.

The method of the present invention is particularly useful for the prevention and treatment of sunburn and other skin damage resulting from exposure to ultraviolet radiation. Glutaredoxin, alone or with other active ingredients can thus be added to dermatological creams and emollients as well as to commercial sunscreens to enhance their sun protection activity, or to creams used to treat sunburn or burns produced by therapeutical radiation used to treat cancer.

It is also worth noting that free radicals seem to be involved in some specific skin diseases, like vitiligo, and these diseases may respond well to treatment with glutaredoxin.

Having described the invention with reference to particular compositions, theories of effectiveness, it will be apparent to those of skill in the art that it is not intended that the invention be limited by such illustrative embodiments or mechanisms, and that modifications can be made without departing from the scope or spirit of the invention, as defined by the appended claims. It is intended that all modifications and variations be included within the scope of the invention. The claims are meant to cover the claimed components and steps in any sequence which is effective to meet the objectives there intended, unless the context specifically indicates the contrary.

We claim:

1. A method for the treatment of skin damage induced by exposure to solar or other type of radiation or to stressors leading to the formation of active oxygen species which consists of applying a composition containing glutaredoxin in a dermatologically acceptable carrier to the affected skin area.

2. A method for the treatment of skin damage induced by exposure to solar or other type of radiation or to stressors leading to the formation of active oxygen species which consists of applying a composition containing glutaredoxin and reduced glutathione in a dermatologically acceptable carrier to the affected skin area.

3. A method in accordance with claims 1 or 2, wherein said composition further comprises one or more additional ingredients selected from the group consisting of tocotrienols, vitamin E, ascorbic acid, superoxide dismutase, catalase, astaxanthin, lycopene.
